(19) United States
(12) Patent Application Publication
(10) Pub. No.: US 2022/0194952 A1
(43) Pub. Date: Jun. 23, 2022

(54) TETRAPYRROLIC CONJUGATES AND USES THEREOF FOR IMAGING

(71) Applicants: Health Research, Inc., Buffalo, NY (US); The Research Foundation for The State University of New York, Amherst, NY (US)

(72) Inventors: Ravindra K. PANDEY, East Amherst, NY (US); Mykhaylo DUKH, Buffalo, NY (US); Aimee MARKO, Buffalo, NY (US); Munawwar SAJJAD, Amherst, NY (US)

(21) Appl. No.: 17/442,905

(22) PCT Filed: Mar. 25, 2020
(86) PCT No.: PCT/US2020/024719

§ 371 Date: Sep. 24, 2021

Related U.S. Application Data
(60) Provisional application No. 62/823,411, filed on Mar. 25, 2019.

(51) Int. Cl. C07D 487/22; A61K 49/00; A61K 41/00

(52) U.S. Cl. CPC C07D 487/22; A61K 41/0071; A61K 49/0036

(57) ABSTRACT

Compounds for tumor imaging (e g , magnetic resonance (MR) and fluorescence) that may be used in combination with other methods to treat an individual having or suspected of having cancer (e.g., various forms of cancer, such as, for example, solid tumors). Compounds may have the following structure: (I) or a salt, a partial salt, a hydrate, a polymorph, an isomer (e.g., a structural or stereoisomer), or a mixture thereof, where R′ is an aryl group or heteroaryl group having a halogen group (e.g., I or ¹²⁴I), X is chosen from O, S, or NH, n is 1-6 (e.g., 1, 2, 3, 4, 5, or 6), the dotted carbon is chiral and is R or S.

### CROSS REFERENCE TO RELATED APPLICATIONS

This application claims priority to U.S. Provisional Application No. 62/823,411, filed on Mar. 25, 2019, the disclosure of which is incorporated herein.

### STATEMENT REGARDING FEDERALLY FUNDED RESEARCH

This invention was made with government support under Grant No. CA127369 awarded by the National Institutes of Health. The government has certain rights in the invention.

### BACKGROUND OF THE DISCLOSURE

Positron emission tomography (PET) is especially useful in the context of cancer diagnosis because it can detect both primary tumors and metastasis that may not be visualized by other imaging techniques. It is also being increasingly used not only as a cancer diagnostic tool, but also to help physicians design the most beneficial therapies. For example, it may be used to assess response to chemotherapy, and is very accurate in assessing the spread of malignant tumors. PET is also used to detect recurrent brain tumors and cancers of the lung, colon, breast, lymph nodes, skin, and other organs.

It has been previously shown that certain tumor-avid porphyrin-based compounds, e.g., methyl 3-[1′-(m-iodo benzyloxy)ethyl]-3-devinyl-pyropheophorbide-a (FIG. 13, Compound 2) can be used for fluorescence imaging and photodynamic therapy. Similar to most of the pyropheophorbide-a analogs, compound 2 exhibits long wavelength absorption at 660 nm, and excitation of the peak at that wavelength exhibits broad emission bands at 670 nm (strong) and 720 nm (weak). A significant part of the strong band observed at 670 nm overlaps the absorption band of the compound. Therefore, the weak band at 720 nm is used for fluorescence imaging of cancer. It works well for imaging superficial tumors, but it is not an ideal candidate for imaging large and deeply seated tumors.

### SUMMARY OF THE DISCLOSURE

This disclosure provides compounds for tumor imaging (e.g., magnetic resonance (MR) and fluorescence) that may be used in combination with other methods to treat an individual having or suspected of having cancer (e.g., various forms of cancer, such as, for example, solid tumors).

In an aspect, the present disclosure provides compounds. The compounds of the present disclosure can be made by methods disclosed herein. The compounds can be used as is described herein. A compound may be referred to as a porphyrin.

A compound can comprise various tetrapyrroles (e.g., tetrapyrrole groups). A tetrapyrrole group may be derived from a tetrapyrrole. In an example, a tetrapyrrole (e.g., tetrapyrrole group) is tumor-avid tetrapyrrole. Non-limiting illustrative examples of tetrapyrroles (e.g., tetrapyrrole groups) include porphyrins and derivatives and analogs thereof, and groups derived therefrom.

In an aspect, the present disclosure provides compositions comprising one or more compound of the present disclosure. The compositions may comprise one or more pharmaceutically acceptable carrier.

In various examples, compounds and compositions of the present disclosure exhibit low or no skin phototoxicity issues following administration to an individual in need of treatment.

In an aspect, the present disclosure provides uses of compounds of the present disclosure. The compounds may be used as imaging agents (e.g., PET imaging agents). In various examples, the present disclosure provides methods that use one or more compounds of the present disclosure. Examples of methods include, but are not limited to, methods of imaging an individual (or a portion thereof) and methods of imaging and treating an individual.

In an aspect, the present disclosure provides products, (e.g., articles of manufacture. Non-limiting examples include, such as, for example, kits. Kits may comprise pharmaceutical preparations containing any one or any combination of the compounds described herein. In an example, the instant disclosure includes a closed or sealed package that contains the pharmaceutical preparation. In certain examples, the package can comprise one or more closed or sealed vials, bottles, blister (bubble) packs, or any other suitable packaging for the sale, or distribution, or use of the pharmaceutical compounds and compositions comprising them. The printed material can include printed information. The printed information may be provided on a label, or on a paper insert, or printed on the packaging material itself. The printed information can include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material can include an indication that the pharmaceutical composition and/or any other agent provided with it is for treatment of cancer and/or any disorder associated with cancer. In examples, the product includes a label describing the contents of the container and providing indications and/or instructions regarding use of the contents of the container to treat any cancer.

### BRIEF DESCRIPTION OF THE FIGURES

For a fuller understanding of the nature and objects of the disclosure, reference may be made to the following detailed description taken in conjunction with the accompanying figures.

FIG. 1 shows extending the isocyclic ring red-shifts absorbance characteristics. Absorbance of 2 and 4 was measured using different concentrations in methanol on UV-VIS and indicates far-red peaks at 660 nm and 785 nm, with extinction coefficients ε₆₆₅=47,500 L·mol⁻¹ cm⁻¹ and ε₇₈₅=18,100 L·mol⁻¹ cm⁻¹ respectively.

FIG. 2 shows fluorescence characteristics of both the compounds (2 & 4) were measured in methanol. Both solutions were irradiated at 532 nm. The results indicate that replacing the fused isocyclic ring with a cyclohexenone ring reduces its fluorescence efficiency significantly.

FIG. 3 shows (A) fluorescence spectrum of 4 in deaerated PhCN excited at 532 nm. (B) Fluorescence decay curve of 4 observed at 787 nm. Excitation wavelength: 651 nm.

FIG. 4 shows phosphorescence of various photosensitizers. The photosensitizers, e. g., HPPH (control), 2 and 4 were individually dissolved in methanol (concentration: 5 μM) in polystyrene cuvettes and excited at 532 nm using a pulsed laser. Phosphorescence of singlet oxygen was detected at 1270 nm. The results indicate that replacing the five-member fused ring in compound 2 with a cyclohexanone ring 4 reduces singlet oxygen quantum yield.

FIG. 5 shows near-IR (NIR) emission spectra of (A) C₆₀ and (B) 4 in O₂-saturated C₆D₆. Excitation wavelength: 532 nm.

FIG. 6 shows (a) transient absorption spectra of 4 after nanosecond laser excitation (λ=532 nm) at 3, 50 and 150 μs. The decay time profiles at 580 nm in (b) argon- and (c) air-saturated PhCN. The single-exponential fitting curves are shown as gray lines.

FIG. 7 shows photobleaching studies. Photobleaching was performed in quartz cuvettes on a 5 μM solution of 2 and a 25 μM solution of 4 in 17% BCS. The higher molarity was chosen to approximate the absorbance of 2 at 5 μM. A dye laser was used at dose rate 75 mW/cm², and excitation wavelength for 2 was 660 nm and for 4 was 785 nm. Compound 4 showed negligible photobleaching while 2 reached the experimental endpoint of 20%. This data correlates with the finding of low singlet oxygen production of compound 2.

FIG. 8 shows (A) in vitro cytotoxicity assays indicate that 2 shows light dose-dependent cytotoxicity, while compound 4 did not produce any significant cell kill evaluated at variable drug concentrations, and at a fixed light dose (1.0 J/cm²). (B) In vitro cytotoxicity assays of 2 indicate toxic effects only at high concentrations. Drug concentrations were increased 10× (0.25 to 32.0 μM) and a significant cytotoxicity was observed only at high concentrations at the highest light dose.

FIG. 9 shows comparative in vivo tumor fluorescence of PS 2 & 4 (BALB/c mice bearing Colon26 tumors (3 mice/group/time point), dose: 0.47 μmol/kg) at variable time points (for details, see Example 1).

FIG. 10 shows comparative in vivo PDT efficacy of PSs 2 and 4 containing an isocyclic ring or a cyclohexanone ring system, respectively, in BALB/c mice bearing Colon26 tumors (5 mice/group). PS 2 at a dose of 1 μmol/kg showed 80% cure, whereas PS 4 at the same dose had no tumor cure. At higher drug doses, no significant long-term PDT efficacy was observed. Tumors were exposed to light (135 J/cm², 75 mW/cm²) at 24 h (hour) postinjection (optimal time for tumor uptake for both the PSs).

FIG. 11 shows Positron Emission Tomography (PET) of 2a (top) and 6 (bottom) formulated in ethanol/PBS at an imaging dose of 25 μCi/mouse (3 BALB/c mice bearing Colon26 tumors/group) produced maximum tumor uptake at 24 hours (see FIG. 12), but best contrasts were observed at 48 and 72 h (hour) postinjection.

FIG. 12 shows in vivo biodistribution of ¹²⁴I-compounds 2a and 6 in BALB/c mice bearing Colon26 tumors (3 mice/PS/time point), the ¹²⁴I-analogs were formulated in ethanol/PBS and injected (25 μCi/mouse) intravenously. Compared to 2a compound 6 containing a cyclohexenone ring showed a significantly higher uptake in tumor than other organs. From left to right, each group is 24 hours 2, 48 hours 2, 72 hours 2, 24 hours 6, 48 hours 6, and 72 hours 6.

FIG. 13 shows synthesis of methyl 3-[1′-(m-iodobenzyloxy)ethyl]-3-devinyl pyropheophorbide (2) and the related analog methyl 3-[1′-(m-iodobenzyloxy)ethyl]-3-devinyl-13³-keto-13²-methoxycyclohexene (4), with a fused cyclohexenone ring system. Synthesis of the radioactive analogs of both compounds proceeds as in steps d and e.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step may be made without departing from the scope of the disclosure.

Ranges of values are disclosed herein. The ranges set out an example of a lower limit value and an example of an upper limit value. Unless otherwise stated, the ranges include all values to the magnitude of the smallest value (either lower limit value or upper limit value) and ranges between the values of the stated range. All ranges provided herein include all values that fall within the ranges to the tenth decimal place, unless indicated otherwise.

The present disclosure provides compounds and uses of the compounds. For example, the compounds are used as PET imaging agents.

As used herein, unless otherwise indicated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent radicals, such as, for example, divalent radicals, trivalent radicals, and the like). Illustrative examples of groups include, but are not limited to:

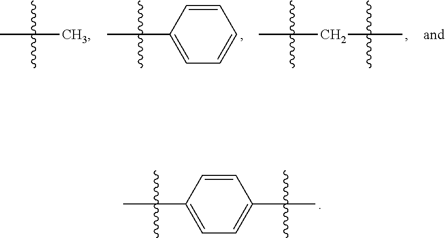

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is a C₁ to C₁₂ alkyl group, including all integer numbers of carbons and ranges of numbers of carbons therebetween (C₁, C₂, C₃, C₄, C₅, C₆, C₇, C₈, C₉, C₁₀, C₁₁, or C₁₂). An alkyl group may be unsubstituted or substituted with one or more substituent. Examples of substituents include, but are not limited to, various substituents such as, for example, halogens (—F, —Cl, —Br, and —I), aliphatic groups (e.g., alkyl groups, alkenyl groups, and alkynyl groups), aryl groups, alkoxide groups, carboxylate groups, carboxylic acids, ether groups, and the like, and combinations thereof.

As used herein, unless otherwise indicated, the term “heteroalkyl group” refers to branched or unbranched saturated hydrocarbon groups comprising at least one heteroatom. Examples of suitable heteroatoms include, but are not limited to, nitrogen, oxygen, sulfur, phosphorus, and halogens. A heteroalkyl group may be unsubstituted or substituted with one or more substituent. Examples of substituents include, but are not limited to, various substituents such as, for example, halogens (—F, —Cl, —Br, and —I), aliphatic groups (e.g., alkenes, alkynes), aryl groups, alkoxides, carboxylates, carboxylic acids, ether groups, and the like, and combinations thereof.

As used herein, unless otherwise indicated, the term “aryl group” refers to C₅ to C₁₂ (C₅, C₆, C₇, C₈, C₉, C₁₀, C₁₁, or C₁₂) aromatic or partially aromatic carbocyclic groups, including all integer numbers of carbons and ranges of numbers of carbons therebetween. An aryl group may be an aromatic group. An aryl groups may comprise polyaryl groups such as, for example, fused ring or biaryl groups. An aryl group may be unsubstituted or substituted with one or more substituent. Examples of substituents include, but are not limited to, substituents such as, for example, halogens (—F, —Cl, —Br, and —I), aliphatic groups (e.g., alkenes, alkynes, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), and fused ring groups (e.g., naphthyl groups and the like).

As used herein, unless otherwise indicated, the term “heteroaryl group” refers to a monovalent monocyclic or polycyclic aromatic group of 5 to 18 (5, 6, 7, 8, 9, 10, 11, 12, 13, 14, 15, 16, 17, or 18) ring atoms or a polycyclic aromatic group, containing one or more ring heteroatoms independently chosen from N, O, S, and combinations thereof, and the remaining ring atoms are C. Heteroaryl groups include polycyclic (e.g., bicyclic) heteroaromatic groups where the heteroatom is N, O, S, or a combination thereof. A heteroaryl group may substituted independently with one or more substituents \. The substituents can be optionally substituted. Examples include, but are not limited to, benzothiophene, furyl, thienyl, pyrrolyl, pyridyl, pyrazinyl, pyrazolyl, pyridazinyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, benzoimidazolyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two fused rings, the heteroaryl groups herein defined may have an unsaturated or partially saturated ring fused with a fully saturated ring.

This disclosure provides compounds suitable for tumor imaging (e.g., magnetic resonance (MR) and fluorescence) that may be used in combination with other methods to treat an individual having or suspected of having cancer (e.g., various forms of cancer, such as, for example, solid tumors).

In an aspect, the present disclosure provides compounds (e.g., tetrapyrrolic compound(s)). The compounds of the present disclosure can be made by methods disclosed herein. The compounds can be used as is described herein. A compound may be referred to as a porphyrin.

A compound can comprise various tetrapyrroles (e.g., tetrapyrrole groups). A tetrapyrrole group can be derived from a tetrapyrrole. In an example, a tetrapyrrole (e.g., tetrapyrrole group) is tumor-avid tetrapyrrole. Non-limiting illustrative examples of tetrapyrroles (e.g., tetrapyrrole groups) include porphyrins and derivatives and analogs thereof, and groups derived therefrom.

In various examples, the compound has the following structure:

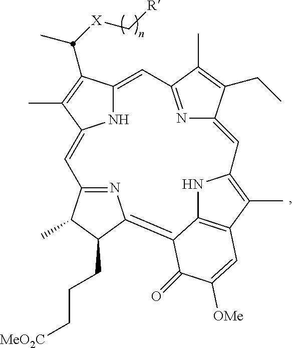

where R′ is an aryl group (e.g., phenyl, napthyl, and the like) or heteroaryl (e.g., pyridinyl, indolyl, furanyl, and the like) having a halogen group (e.g., I or ¹²⁴I) and n is 1-6 (e.g., 1, 2, 3, 4, 5, or 6). X is chosen from O, S, or NH. The dotted carbon is chiral and is R or S.

In various examples, the compound has the following structure:

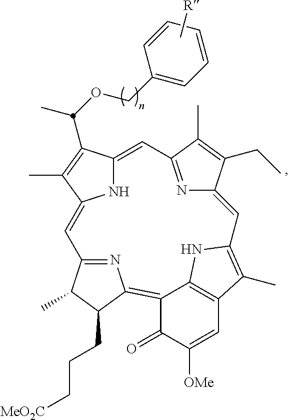

where R″ is a halogen group (e.g., I or ¹²⁴I), n is 1-6 (e.g., 1, 2, 3, 4, 5, or 6), and the dotted carbon is chiral and is R or S.

In various examples, the compound has the following structure:

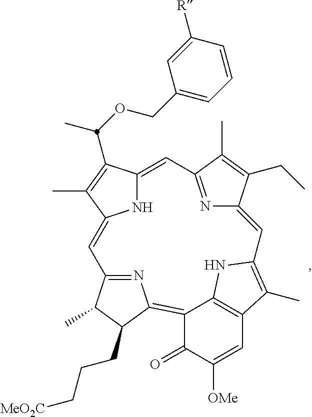

where R″ is a halogen group (e.g., I or ¹²⁴I) and the dotted carbon is chiral and is R or S.

In various examples, the compound has the following structure:

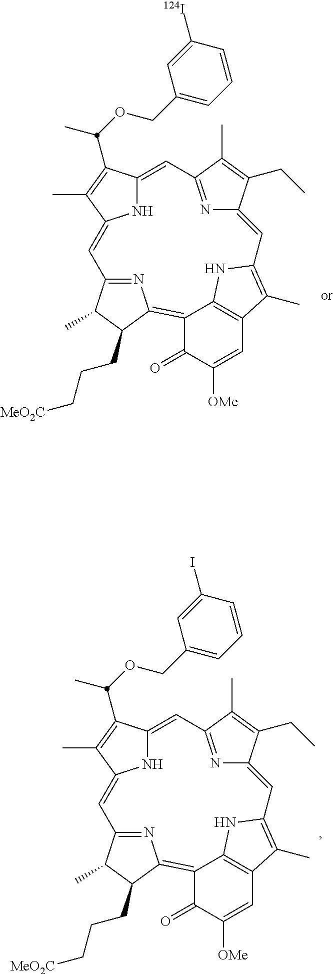 [SMILES: CCC1=C(C)c2cc3[nH]c(cc4nc(c5c6[nH]c(cc1n2)c(C)c6C=C(OC)C5=O)[C@@H](CCCC(=O)OC)[C@@H]4C)c(C)c3[C@H](C)OCc1cccc([124I])c1 ; CCC1=C(C)c2cc3[nH]c(cc4nc(c5c6[nH]c(cc1n2)c(C)c6C=C(OC)C5=O)[C@@H](CCCC(=O)OC)[C@@H]4C)c(C)c3[C@H](C)OCc1cccc(I)c1]

where the dotted carbon is chiral and is R or S.

In various examples, the compound is a salt, a partial salt, a hydrate, a polymorph, an isomer (e.g., a structural or stereoisomer), or a mixture thereof. The compounds may have stereoisomers. For example, the compound is present as a racemic mixture, a single enantiomer, a single diastereomer, mixture of enantiomers, or mixture of diastereomers.

The compounds of the present disclosure include pharmaceutically acceptable derivatives and prodrugs of the compounds of the present disclosure. A compound may be a lyophilized compound (e.g., a lyophilized powder).

Compounds of the present disclosure comprising 124I may have a half-life (e.g., several days) longer than other PET agents known in the art (e.g., PET agents comprising ¹⁸F). Compounds of the present disclosure may exhibit other desirable properties, such as, for example, it can be formulated into a variety of formulating agents (e.g., polymers for biodegradable and non-toxic nanoparticles).

In an aspect, the present disclosure provides compositions comprising one or more compound of the present disclosure. The compositions may comprise one or more pharmaceutically acceptable carrier.

In various examples, compounds and compositions of the present disclosure exhibit low or no skin phototoxicity issues following administration to an individual in need of treatment.

The compositions can include one or more standard pharmaceutically acceptable carriers. The compositions may include solutions, suspensions, emulsions, and solid injectable compositions that are dissolved or suspended in a solvent before use. The injections may be prepared by dissolving, suspending or emulsifying one or more active ingredient(s) (e.g., a compound of the present disclosure) in a diluent. Examples of diluents are distilled water for injection, physiological saline, vegetable oil, alcohol, and a combination thereof. Further, the compositions suitable for injections may comprise stabilizers, solubilizers, suspending agents, emulsifiers, soothing agents, buffers, preservatives, and the like, and combinations thereof. The compositions (e.g., compositions suitable for injection) may be sterilized in the final formulation step or prepared by sterile procedure. The pharmaceutical composition may be formulated into a sterile solid preparation, for example, by freeze-drying, and can be used after sterilized or dissolved in sterile injectable water or other sterile diluent(s) immediately before use. Pharmaceutically-acceptable carriers include, but are not limited to, sugars, such as lactose, glucose and sucrose; starches, such as corn starch and potato starch; cellulose, including sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, and combinations thereof. The composition, if desired, may contain minor amounts of wetting or emulsifying agents, or pH buffering agents. Additional non-limiting examples of pharmaceutically acceptable carriers can be found in: Remington: The Science and Practice of Pharmacy (2005) 21st Edition, Philadelphia, Pa. Lippincott Williams & Wilkins.

In an aspect, the present disclosure provides uses of compounds of the present disclosure. The compounds may be used as imaging agents (e.g., PET imaging agents).

In various examples, the present disclosure provides methods that use one or more compounds of the present disclosure. Examples of methods include, but are not limited to, methods of imaging an individual (or a portion thereof) and methods of imaging and treating an individual.

This disclosure provides methods of treating individuals in need of treatment (e.g., for a hyperproliferative disorder, such as, for example, malignancy (e.g., a malignancy disorder)) comprising administering to an individual a compound or composition of the present disclosure, and imaging the individual or a portion thereof and, after staging the disease, proceeding to appropriate therapy (e.g., surgical, chemotherapeutic, photodynamic, or standard radiation).

The image can be obtained using a techniques known in the art. Non-limiting examples include magnetic resonance imaging and, in some cases, fluorescent imaging.

Methods of the present disclosure can be carried out in or performed on an individual who has been diagnosed with or is suspected of having cancer. A method can also be carried out in individuals who have a relapse or a high risk of relapse after being treated for cancer.

In various examples, a method for detecting the presence of a hyperproliferative tissue in an individual comprises: administering to the individual an effective quantity of one or more compounds and/or one or more compositions of the present disclosure; and imaging the individual or a portion thereof to detect the presence and/or absence of a hyperproliferative tissue(s) in the individual.

In various examples, imaging can be performed using only a single administration of a compound(s) or composition(s) of the present disclosure. In various examples, after a single administration, the target issue can be imaged immediately after administration, after 24 hours, after 48 hours, after 72 hours, and/or after 96 hours.

In various examples, a compound or composition of the present disclosure can be administered in low doses relative to other clinically relevant options known in the art. For example, dosing is half or less of a typical PET agent known in the art. In another example, dosing is a quarter or less of a typical PET agent known in the art. For example, doses can be 50 μCi or less, 100 μCi or less, 150 μCi or less, 200 μCi or less, 500 μCi or less, 750 μCi or less, or 1000 μCi or less.

A method may use one or more lyophilized compound(s) and/or one or more composition(s) comprising one or more lyophilized compound. The lyophilized compound(s) and/or composition(s) may be reconstituted prior (e.g., immediately prior) to administration to the individual and immediately prior to imaging.

Methods of the present disclosure can be used on various individuals. Individuals are also referred to herein as subjects. In various examples, an individual is a human or non-human mammal. Examples of non-human mammals include, but are not limited to, agricultural animals (e.g., farm animals), such as cows, hogs, sheep, and the like, as well as pet, service, or sport animals such as horses, dogs, cats, and the like. Additional non-limiting examples of individuals include rabbits, rats, and mice.

“Hyperproliferative disorders” as used herein refers to conditions and/or disorders sharing as an underlying pathology of excessive cell proliferation caused by unregulated or abnormal cell growth, and include uncontrolled angiogenesis. Examples of hyperproliferative disorders include, but are not limited to, cancers or carcinomas.

Various hyperproliferative tissues can be imaged and/or treated using methods of the present disclosure. Non-limiting examples of hyperproliferative tissues include vascular endothelial tissue, a neovasculature tissue, a neovasculature tissue present in the eye, an abnormal vascular wall of a tumor, a solid tumor, a tumor of the pancreas, a tumor of the bladder, a tumor of a head, a tumor of a neck, a tumor of an eye, a tumor of a gastrointestinal tract, a tumor of a liver, a tumor of a breast, a tumor of a prostate, a tumor of a brain, a tumor of a lung, a nonsolid tumor, malignant cells of one of a hematopoietic tissue and a lymphoid tissue. Combinations of hyperproliferative tissues can be imaged.

In various examples, a method of the present disclosure comprises administering to an individual one or more compound and/or one or more composition of the present disclosure. The compound(s) and/or composition(s) can be delivered to the vascular system of an individual by using, for example, intravascular delivery.

“Irradiating” and “irradiation” as used herein includes exposing an individual to any wavelength of light. Preferably, the irradiating wavelength is selected to match the wavelength(s) that excite the photosensitizing compound (e.g., a compound of the present disclosure). Preferably, the radiation wavelength matches the excitation wavelength of the photosensitizing compound (e.g., compound of the present disclosure) and has low absorption by the non-target tissues of the individual, including blood proteins.

Exposure (e.g., irradiation) may be defined by its coherence (laser) or non-coherence (non-laser), as well as intensity, duration, timing with respect to dosing using a compound of the present disclosure (e.g., a photosensitizing compound), or any combination thereof. The intensity or fluence rate is sufficient for the light to reach the target tissue. In various examples, the radiation energy is provided by an energy source, such as, for example, a laser or cold cathode light source, that is external to the individual, or that is implanted in the individual, or that is introduced into an individual, such as by a catheter, optical fiber or by ingesting the light source in capsule or pill form (e.g., as disclosed in U.S. Pat. No. 6,273,904 (2001), the disclosure of which, with regard to energy sources, is incorporated herein by reference).

In an example, a method is carried out using a single MM scanner and a single dose of the compound(s) and/or composition(s). In another example; a method is carried out using a single dose of the compound(s) and/or composition(s). In another example; a method is carried out using a single dose of the compound(s) and/or composition(s) and no radiation exposure.

In an aspect, the present disclosure provides products (e.g., articles of manufacture). Non-limiting examples include, such as, for example, kits. Kits may comprise pharmaceutical preparations containing any one or any combination of the compounds described herein. In an example, the instant disclosure includes a closed or sealed package that contains the pharmaceutical preparation. In certain examples, the package can comprise one or more closed or sealed vials, bottles, blister (bubble) packs, or any other suitable packaging for the sale, or distribution, or use of the pharmaceutical compounds and compositions comprising them. The printed material can include printed information. The printed information may be provided on a label, or on a paper insert, or printed on the packaging material itself. The printed information can include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material can include an indication that the pharmaceutical composition and/or any other agent provided with it is for treatment of cancer and/or any disorder associated with cancer. In examples, the product includes a label describing the contents of the container and providing indications and/or instructions regarding use of the contents of the container to treat any cancer.

The steps of the methods described in the various examples disclosed herein are sufficient to carry out the methods of the present disclosure. Thus, in an example, a method consists essentially of a combination of the steps of the methods disclosed herein. In another example, a method consists of such steps.

The following Statements describe various non-limiting examples of the present disclosure:
Statement 1. A compound having the following structure:

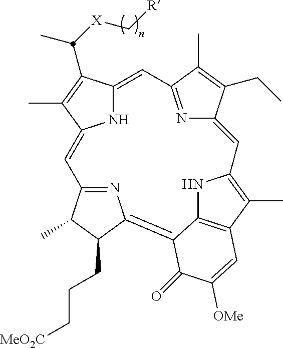

or a salt, a partial salt, a hydrate, a polymorph, an isomer (e.g., a structural or stereoisomer), or a mixture thereof,
where R′ is an aryl group or heteroaryl group having a halogen group (e.g., I or ¹²⁴I), X is chosen from O, S, or NH, n is 1-6 (e.g., 1, 2, 3, 4, 5, or 6), the dotted carbon is chiral and is R or S.
Statement 2. A compound according to Statement 1 having the following structure:

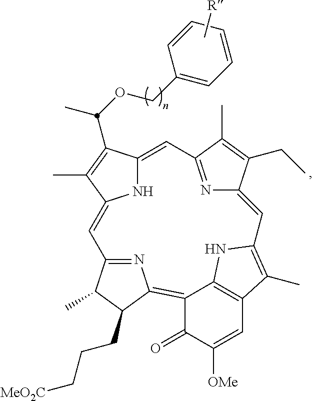

where R″ is a halogen group (e.g., I or ¹²⁴I), n is 1-6 (e.g., 1, 2, 3, 4, 5, or 6), and the dotted carbon is chiral and is R or S.
Statement 3. A compound according to Statement 1 or Statement 2 having the following structure:

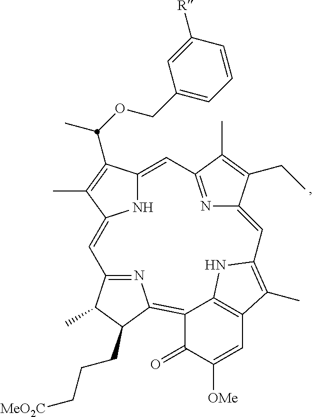

where R″ is a halogen group (e.g., I or ¹²⁴I) and the dotted carbon is chiral and is R or S.
Statement 4. A compound according to any one of the preceding Statements having the following structure:

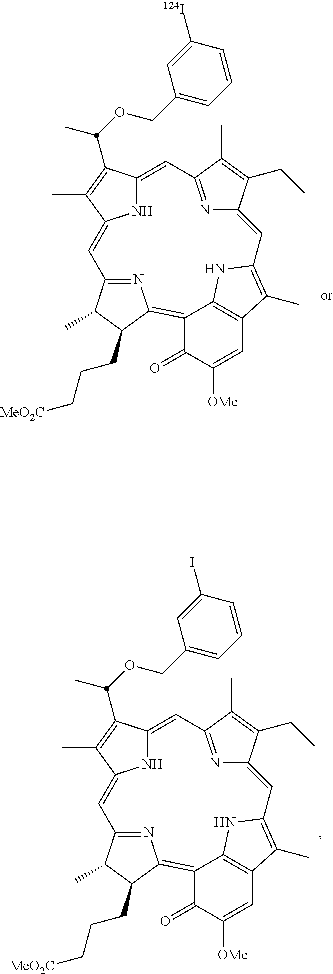 [SMILES: CCC1=C(C)c2cc3[nH]c(cc4nc(c5c6[nH]c(cc1n2)c(C)c6C=C(OC)C5=O)[C@@H](CCCC(=O)OC)[C@@H]4C)c(C)c3[C@H](C)OCc1cccc([124I])c1 ; CCC1=C(C)c2cc3[nH]c(cc4nc(c5c6[nH]c(cc1n2)c(C)c6C=C(OC)C5=O)[C@@H](CCCC(=O)OC)[C@@H]4C)c(C)c3[C@H](C)OCc1cccc(I)c1]

where the dotted carbon is chiral and is R or S.
Statement 5. A composition comprising a compound of any one of the preceding Statements and a pharmaceutically acceptable carrier.
Statement 6. A method of imaging a hyperproliferative tissue in an individual (e.g., a subject), comprising:
   administering to the individual a composition according to Statement 5;
   exposing the individual or a portion thereof with electromagnetic radiation; and imaging the individual or a portion thereof.
Statement 7. A method of Statement 6, where after a single administration of the composition of Statement 5, imaging is performed up to 24 hours later, up to 48 hours later, up to 72 hours later, and/or up to 96 hours later.
Statement 8. A method according to Statement 6 or Statement 7, where the hyperproliferative tissue is vascular endothelial tissue, a neovasculature tissue, a neovasculature tissue present in the eye, an abnormal vascular wall of a tumor, a solid tumor, a tumor of the pancreas, a tumor of the bladder, a tumor of a head, a tumor of a neck, a tumor of an eye, a tumor of a gastrointestinal tract, a tumor of a liver, a tumor of a breast, a tumor of a prostate, a tumor of a brain, a tumor of a lung, a nonsolid tumor, malignant cells of one of a hematopoietic tissue, a lymphoid tissue, or combinations thereof.
Statement 9. A kit, comprising

one of more compound of any one of Statements 1-4 and/or one or more composition according to Statement 5; and

instructions for use of the one or more compound and/or the one or more composition.

The following example is presented to illustrate the present disclosure. It is not intended to be limiting in any matter.

### EXAMPLE 1

This example provides a description of the compounds of the present disclosure, methods of making compounds of the present disclosure, and uses of compounds of the present disclosure.

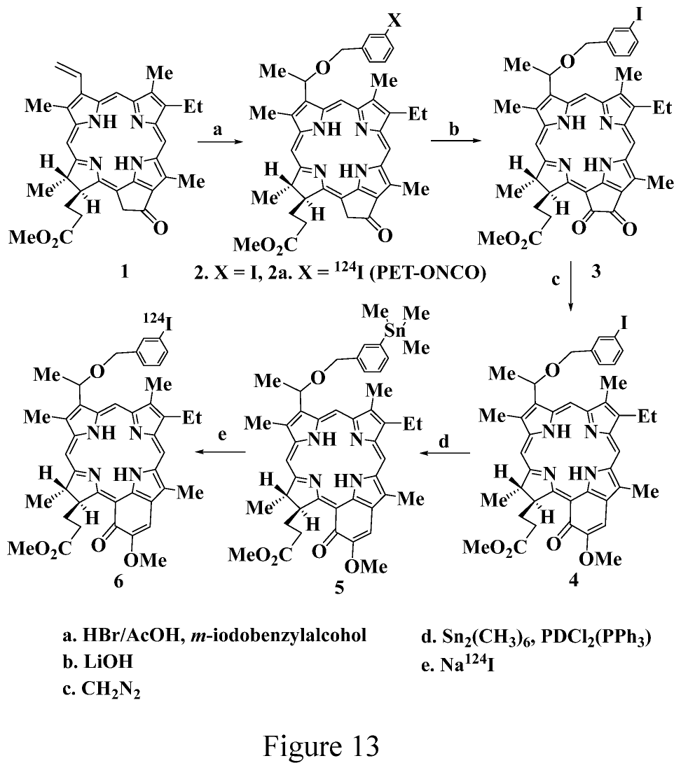

See FIG. 13 for the numbering of the compounds of the present example.

Described is the synthesis, photophysical properties fluorescence imaging and PDT efficacy of the methyl ester analog of 3-(1′-benzyloxy)ethyl-3-devinyl-verdin 4. The PET imaging ability and in vivo biodistribution of the corresponding ¹²⁴I-analog were compared with the well-studied PET-ONCO [3-(1′-m-iodobenzyloxy) ethyl-3-devinyl-pyropheo phorbide-a methyl ester 2a formulated in ethanol/PBS formulation. Interestingly, replacing the five member isocyclic ring of the pyropheophorbide a with the cyclohexenone ring system enhanced its tumor-imaging ability with improved tumor contrast. Interestingly, the new chromophore showed reduced in vivo fluorescence and PDT efficacy, and resulted in the discovery of an excellent porphyrin-based PET imaging agent for cancer imaging and monitoring the disease. Compound 6 containing an ¹²⁴I-radionuclide with the longer half-life (4 days) positron emitter isotope provides new opportunities in molecular imaging.

Several compounds known in the art show significant PDT efficacy with desired photophysical properties, but also produced high uptake in spleen, kidney and liver, which of course cleared with time, but were not suitable candidates for whole body PET imaging.

It has been previously shown that on treatment of 13²-oxo-bacteriopyropheophorbide-a methyl ester with excess diazomethane resulted in the formation of bacterioverdins containing a fused six-membered methoxy-substituted cyclohexenone (verdin) ring system, and the resulting candidates as an isomeric mixture exhibited the long wavelength absorption in the IR region (865-900 nm). These compounds in isomerically pure forms produced desired photophysical properties, but were difficult to separate in large quantity, which hampered there further development. To investigate the impact of chlorin vs. bacteriochlorin analogs, a pyropheophorbide system of methyl 3-(1′-m-iodobenzyloxy) ethyl-3-devinyl-13²-oxo-pyropheophorbide a (FIG. 13, Compound 3) as a substrate was investigated. Surprisingly, in contrast to bacteriochlorins, reaction of the diketochlorin 3 with diazomethane did not produce an isomeric mixture, but gave 4 methyl 3-devinyl-3-[1′-(m-iodobenzyloxy)ethyl]-13³-keto-13²-methoxycyclohexene (verdin) as a sole product in excellent yield with long wavelength absorption at 785 nm.

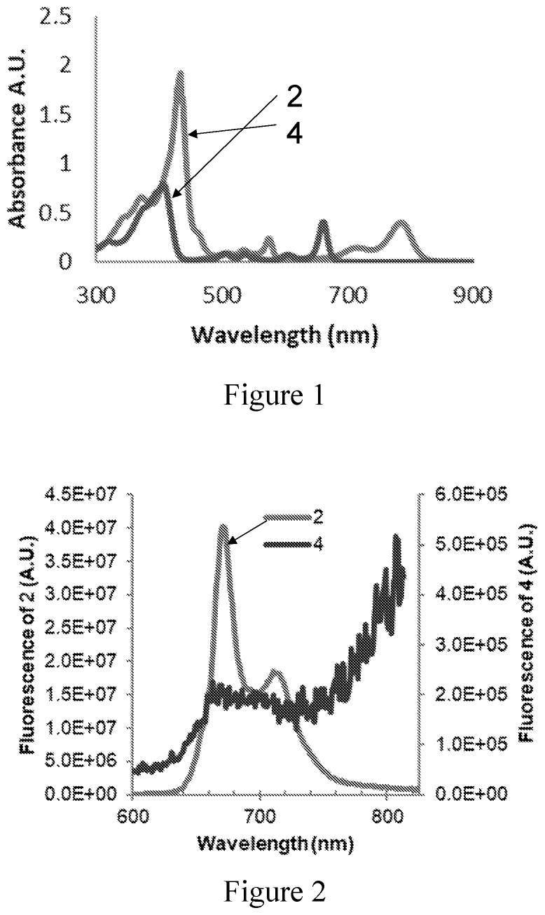

Photophysical properties: The absorbance spectra of chlorins 2 and 4 are shown in FIG. 1. Interestingly, compared to compound 2 containing a five-member isocyclic ring, the related analog bearing a six-member cyclohexenone ring system showed almost 125 nm red shift to its long wavelength absorption peak.

The fluorescence spectra of chlorins 2 and 4 obtained on excitation at 532 nm in methanol are depicted in FIG. 2. Compared to chlorin 2 which exhibited two fluorescence bands at 670 nm (strong) and 720 nm (weak), chlorin 4 with extended ring system showed one very weak emission peak at 810 nm. This peak became more pronounced using dichloromethane as a solvent (not shown).

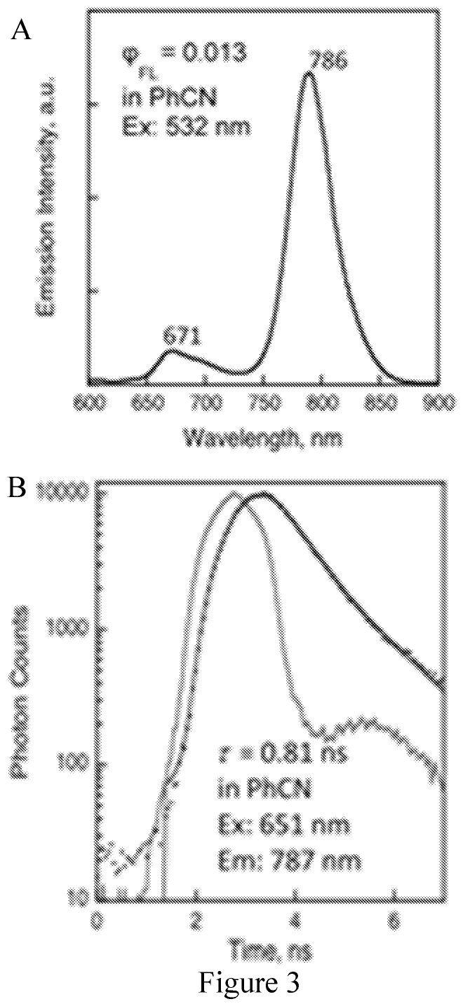

To confirm fluorescence capability, the dry powder of 4 was dissolved in benzonitrile (PhCN), shown in FIG. 3. Fluorescence quantum yield was calculated to be Φ=0.013 with a lifetime of 0.81 ns, which is comparable to fluorescence of Near-IR cyanine dyes and certain core-modified porphyrins, though somewhat lower than some chlorins and bacteriochlorins (0.045 to 0.23) in methanol.

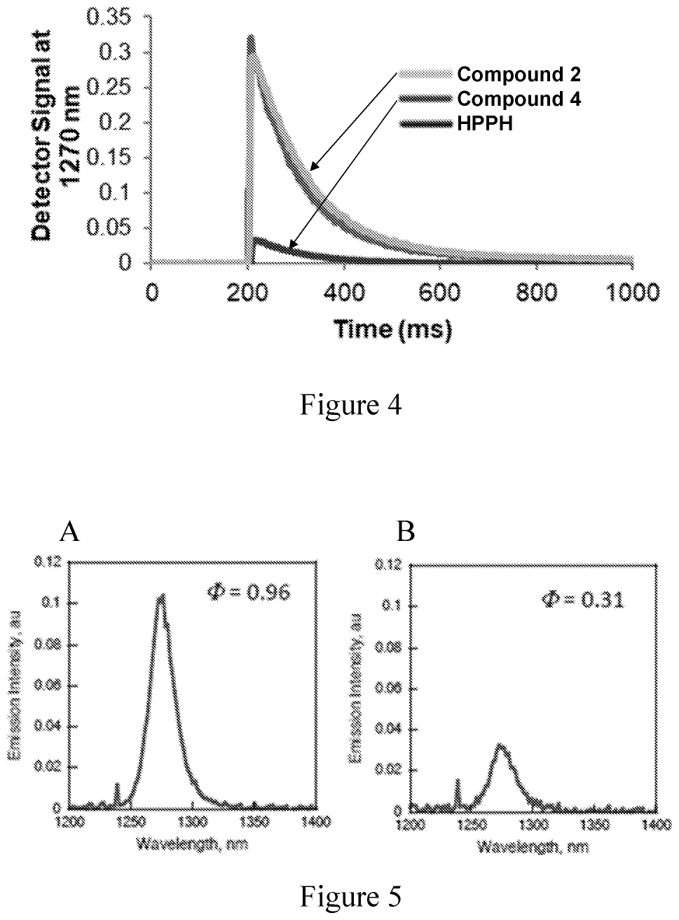

Before evaluating the imaging and photosensitizing potential of the chlorin 4 with extended ring system, its singlet oxygen producing ability was compared with the related chlorin 2. In brief, both compounds were individually dissolved in methanol in equi-absorbant (at 532 nm) concentrations near 5 μM and excited at 532 nm using a pulsed laser. The phosphorescence of singlet oxygen was detected at 1270 nm. From the results summarized in FIG. 4, it can be seen that compared to chlorin 2 with a five-member isocyclic ring, the related analog 4 with an extended ring system had remarkably lower singlet oxygen producing ability. Singlet oxygen quantum yields were φ=0.29 and φ=0.02 respectively, compared to HPPH φ=0.45.

After monitoring absorption characteristics in several organic and aqueous solvents, it was hypothesized that the aggregation state of 4 could have contributed in decreasing the fluorescence and singlet oxygen quantum yields. The quantum yield of singlet oxygen from 4 was Φ_{O2}=0.31 determined by the comparison of the emission intensity at 1270 nm as compared with C₆₀ used as a standard (Φ_{O2}=0.96) (FIG. 5).

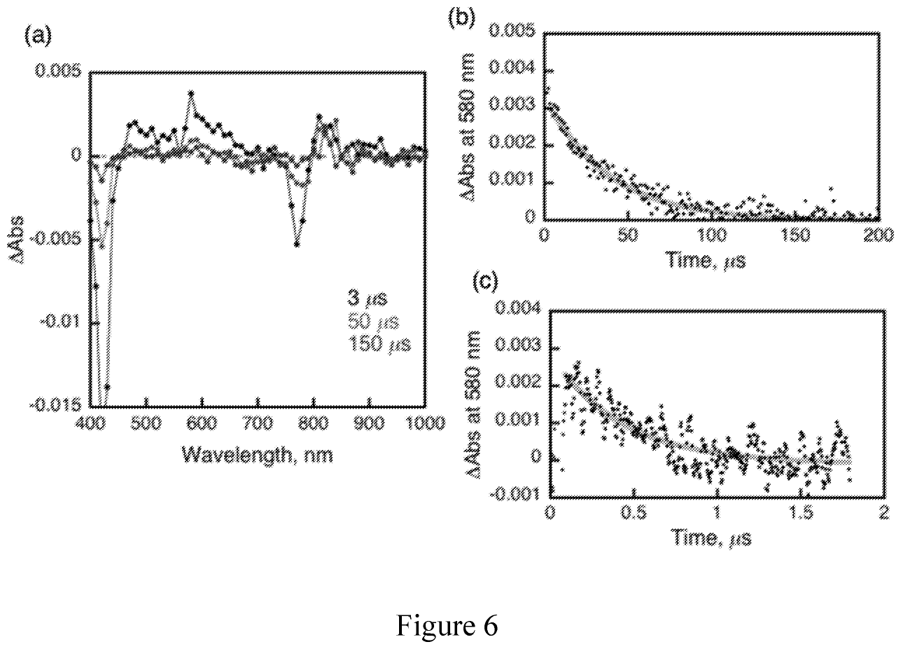

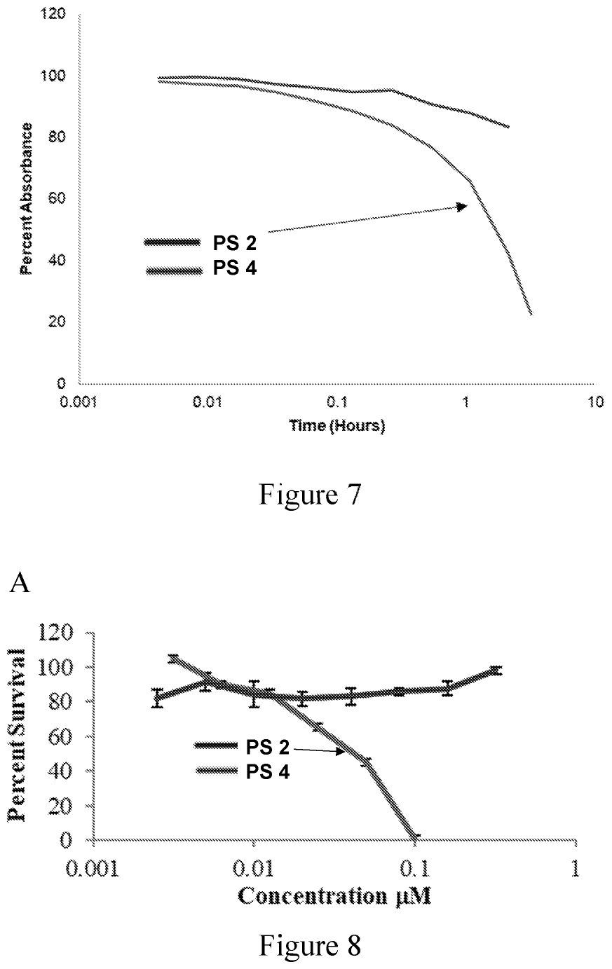

Singlet oxygen is generated by energy transfer from the triplet excited state of 4 to molecular oxygen (O₂). The triplet excited state was spectroscopically detected by laser flash transient absorption measurements as shown in FIG. 6a. The triplet-triplet (T-T) transient absorption spectrum due to the triplet excited state of 4 was observed in deaerated PhCN at 3 μs after nanosecond laser excitation at 532 nm. The decay profile of T-T absorption was obeyed with the single exponential curve (FIG. 6b). The lifetime was determined to be τ=38 μs. The decay rate constant was significantly increased in the presence of dissolved O₂ (2.3×10⁶ s⁻¹) (FIG. 6c). The energy transfer rate constant was determined to be 1.4×10⁹M⁻¹ s⁻¹, indicating the efficient energy transfer occurred between the triplet excited state of 4 and O₂ to for singlet oxygen generation. It was observed that the singlet oxygen producing ability of compound 4 was modest, but only in a nonpolar solvent, and therefore, these results may not correlate with the ROS production either in cell culture or in vivo environment.

To further confirm the difference in singlet oxygen producing ability of chlorins 2 and 4 under physiological conditions, the PSs were dissolved in albumin-containing aqueous solvents, these formulations were subjected for in vitro PDT efficacy and photobleaching studies.

In photodynamic therapy, photobleaching is the photochemical alteration of a chromophore or a fluorophore molecule such that could either permanently is unable to fluoresce or alter its fluorescence characteristics. This is caused by cleaving of the covalent bonds or non-specific reaction between the reactive oxygen species (e.g., singlet oxygen) and the molecule. These nonreactive species formed by conversion of the molecular oxygen present in tumor to singlet oxygen by exposing the photosensitizer with light at an appropriate wavelength. The rate of photobleaching of the PS should depend (a) availability of oxygen, (b) photostability of PS and finally (c) the singlet oxygen (or other reactive oxygen species) producing ability of the PS.

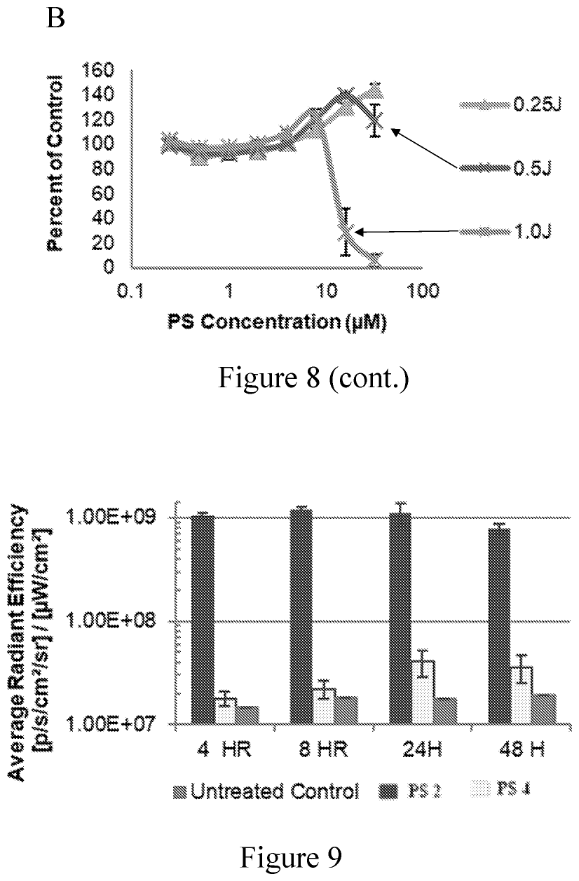

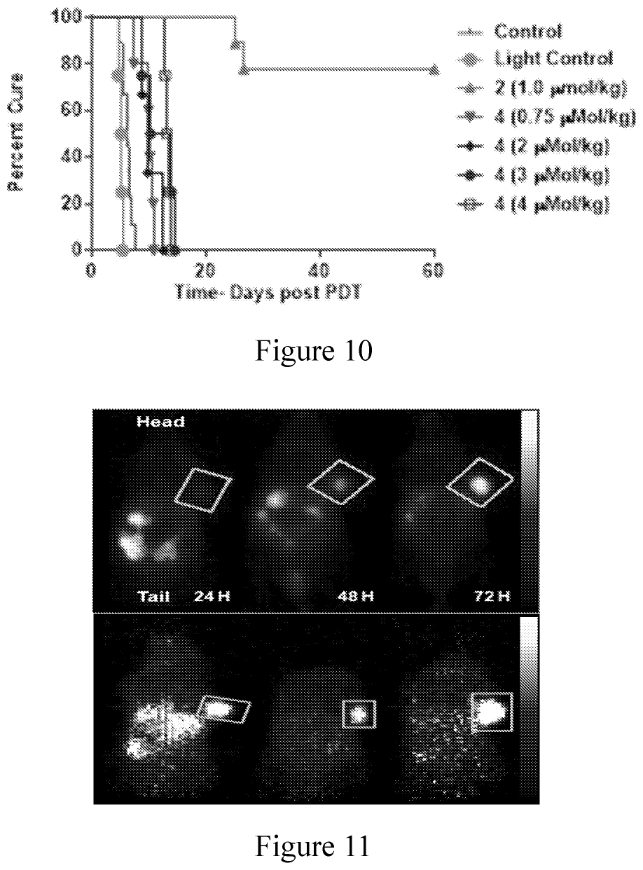

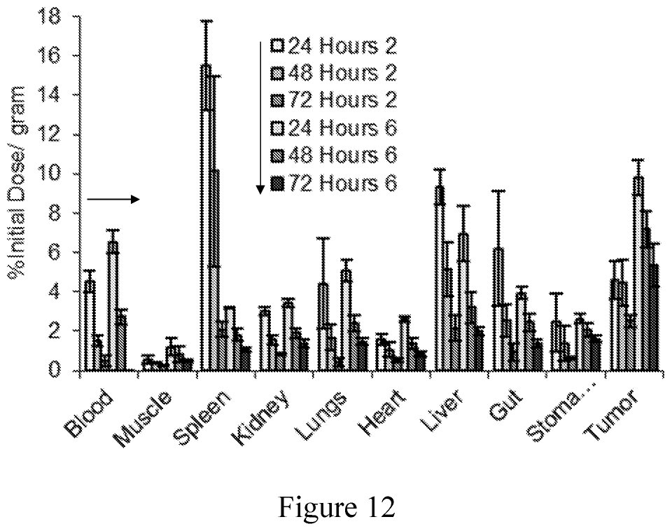

To investigate the photobleaching characteristics of the photosensitizers, compounds 2 and 4 in equimolar concentrations (5 μM) were taken in quartz cuvettes in 17% bovine calf serum. Dye lasers with appropriate wavelength (2: 660 nm and 4: 785 nm) were used at a dose rate of 75 mW/cm². Both the compounds in formulated forms were irradiated with light and the absorption spectra at their long wavelength absorptions were measured with time. The decay in absorbance was plotted against the time of light exposure. From the results summarized in FIG. 6, it is evident that compound 4 showed negligible photobleaching in 17% BCS. Compound 2 showed higher photobleaching capability, as 80% of it was photobleached by three hours. These data suggest that the difference in rate of photobleaching between the two compounds is due to their difference in singlet oxygen producing ability. These results also correlate with the findings summarized in FIGS. 8A and 8B respectively.

Assessment of cell growth following treatment with compound solutions alone and after light exposure was determined by a colorimetric assay that measured the reduction of yellow 3-(4,5-dimethythiazol-2-yl)-2,5-diphenyl tetrazolium bromide (MTT) by mitochondrial succinate dehydrogenase.

Briefly cells were plated into 96 well plates, allowed to adhere to the plate and are treated with a range of compound concentrations for various lengths of time. The light source consisted of dye lasers (375; Spectra-Physics, Mt. View, Calif.) pumped by an argon-ion laser (either 171 or 2080; Spectra-Physics). The dye lasers were tuned to the appropriate wavelength for the compound being tested. Total light doses were ranged from 0.125 to 1.0 J/cm² at a fluence rate of 3.2 mW/cm². At 48 hours post PDT treatment, MTT was added. The solubilized formazan product was measured spectro-photometrically using a BIOTEK EL-800 plate reader. Data were processed using GEN5 software. A medium blank value was subtracted from all samples and Optical Density (O.D.) values of treated cells were divided by mean O.D. value of untreated cells for each light dose).

As can be seen, the in vitro cytotoxicity results indicate toxic effects of PS only at high concentrations, which are usual treatment parameters for most of the active PSs. Colon26 cells were plated and allowed to adhere for 4 hours, then the photosensitizer was added and incubated at 37° C. for 24 hours. Plates were irradiated with 789 nm light from a dye laser at 3.2 mW/cm² for a total dose of 0.25 to 1.0 Joule/cm².

Before evaluating in vivo PDT efficacy of PS 4 by exposing the tumors with light, the time point for optimal tumor uptake of PS was determined by whole body fluorescence imaging of tumored mice at various time points after injecting the PS.

The multi-spectral imaging system, IVIS Spectrum (Perkin-Elmer) along with Living Image (image acquisition and analysis software) was used. IVIS Spectrum has the capability to use either trans-illumination (from the bottom) or epi-illumination (from the top) to illuminate in vivo fluorescent sources. The instrument is equipped with 10 narrow band excitation filters (30 nm bandwidth) and 18 narrow band emission filters (20 nm bandwidth) that assist in significantly reducing auto fluorescence by the spectral scanning of filters and the use of spectral unmixing algorithms. Regions of interest (ROI) were defined for areas of compound accumulation (tumor, liver, skin) and total and average signal within the region were recorded. Fluorescent intensity is expressed as the total radiant efficiency ([p/s]/[μW/cm²]. Results are expressed as mean total radiant efficiency of three mice±SD.

For determining the in vivo imaging and therapeutic potential of 2, BALB/c mice (5-10 mice/group) bearing Colon26 tumors were used. In brief, 1×10⁶ Colon26 cells were injected in mice subcutaneously. The tumors were left to grow for 1 week until they achieve the desired size (3-5 mm) for treatment. The photosensitizer was injected intravenously following the animal protocol approved by RPCI IACUC committee. Irradiance occurred 24 hours after i. v. injection; light dose was 135 J/cm² at a dose rate of 75 mW/cm² using the near-IR wavelengths of interest: 665 nm or 789 nm.

Two axes of the tumor (L, longest axis; W, shortest axis) were measured in millimeters with the aid of a Vernier caliper. Tumor weight (mg) was estimated as a formula: tumor weight=1/2 (L×W2). Tumor measurements were taken daily for the first 10 days and at least three tine a week for the first 4 weeks of post therapy and twice a week thereafter. Endpoints were 400 mm³ tumor volume or 60 days.

The PDT efficacy of 4 was seen to be far lower than that of 2, and a direct correlation between the concentration of the drug present in tumor and long-term PDT efficacy was observed.

The in vivo fluorescence imaging data obtained from the iodinated compounds 2 and 4 (non-radioactive analogs) indicated optimal uptake in tumor at 24 h, but better tumor contrasts were observed at 48 and 72 h post-injection (radioactive analogs), which could be due to faster clearance of the compound from other organs. To investigate the utility of this compound for imaging cancer by PET, the non-radioactive compounds were converted to the corresponding I-124 analogs by following the reaction sequence illustrated in FIG. 13. The purity of the desired radioactive compounds (2a and 6) was assessed by analytical HPLC. Compounds were resuspended in 10% ethanol saline, and ˜50 μCi were injected per mouse.

PET imaging indicated that maximal uptake occurred at 24 hours, and the greatest contrast was seen at 72 hours; both compounds were retained well in the tumor. In vivo biodistribution of the compounds in BALB/c mice implanted with Colon26 tumors indicated the superiority of compound 6 (I-124 analog of 4) over the I-124 analog 2a (¹²⁴I-analog of 2) in PET imaging of cancer due to its higher uptake in tumor compared to other organs. In contrast, the I-124 analog of 2 (2a) in ethanol/PBS formulation showed a significantly high uptake in spleen. Thus, compound 6 (with tumor uptake >9% of the injected dose) provides an opportunity to explore its utility in imaging a variety of cancer types, and monitoring the treatment response.

The interesting findings discussed in this report concludes that the tumor-specificity of porphyrin-based compounds can be influenced not only by replacing the sub stituents at the periphery of the molecule, but also by the nature of the exocyclic ring present in the system. The iodinated compound bearing a fused cyclohexenone ring and an ¹²⁴I-isotope provides an opportunity to image a variety of tumors where the most commonly used PET agents (e.g., ¹⁸F-FDG and other PET agents) show limitations. The longer half-life of ¹²⁴I-over ¹⁸F-(110 min) or ¹¹C-(11 min) in long-distance transportation, which should also make PET more practical and economical.

Experimental.

Chemistry Methods: All reactions were carried out in heat gun-dried glassware under an atmosphere of nitrogen and magnetic stirring. Thin layer chromatography (TLC) was done on pre-coated silica gel sheets (layer thickness 0.2 mm). Column chromatography was performed either using silica gel 60 (70-230 mesh) or neutral alumina grade III. In some cases preparative TLC was used for the purification of compounds. Purity of the compounds was ascertained by TLC and HPLC analysis. All compounds including the intermediates were >95% pure. UV-visible spectra were recorded on FT UV-visible spectrophotometer using methanol as a solvent. Mass spectrometry analyses were performed at the Mass Spectrometry Facility, University at Buffalo, N.Y. Compounds 1 and 2 were synthesized by following the methodologies previously described.

Synthesis of 3-(1′-m-iodobenzyloxy)ethyl-3-devinyl-13¹,13²-dioxopyropheophorbide-a methyl ester (3): The reaction was performed according to the method described in the literature. Compound 2 (100 mg) was dissolved in THF (33 mL) and suspension of LiOH (170 mg) in H₂O (3 mL) was added to the solution. The reaction mixture was stirred overnight at room temperature then poured into H₂O (170 mL) containing CH₃COOH (1.7 mL). The product was extracted with DCM-THF mixture (1:1, 200 mL). Organic extract was washed with water (5×200 mL, until pH of the washing water was >6.5), dried over Na₂SO₄ and briefly (10 min.) treated with excess ethereal diazomethane; the solvents were evaporated in vacuum. The product was purified by prep-TLC (3 plates) using 3% Acetone in DCM as eluent. 43 mg of 3 was obtained. ¹H NMR (400 MHz, CDCl₃, δ ppm^{#}): 10.28 (s, 1H, meso-H), 9.90/9.89 (s, 1H, meso-H), 9.01 (s, 1H, meso-H), 7.74 (m, 1H, phenyl-H), 7.644/7.640 (m, 1H, phenyl-H), 7.31 (m, 1H, phenyl-H), 7.065/7.061 (dd, 1H, J=7.7, 7.7 Hz, phenyl-H), 6.08 (q, 1H, J=6.7 Hz, 3¹-H), 5.20 (m, 1H, 17-H), 4.72 (d, 1H, J=11.7 Hz, —OCHH), 4.70 (q, 1H, J=7.4 Hz, 18-H), 4.614/4.608 (d, 1H, J=11.7 Hz, —OCHH), 3.86/3.85 (s, 3H, ring-CH₃), 3.82 (q, 2H, J=7.7 Hz, 8-CH₂CH₃), 3.59 (s, 3H, —CO₂CH₃), 3.49/3.48 (s, 3H, ring-CH₃), 3.32/3.31 (s, 3H, ring-CH₃), 2.81, 2.69, ˜2.36, ˜2.32 (each m, 4H, 17-CH₂CH₂CO₂CH₃), 2.232/2.227 (d, 3H, J=6.7 Hz, 3¹-CH₃), 1.90/1.89 (d, 3H, J=7.4 Hz, 18-CH₃), 1.77 (t, 3H, J=7.7 Hz, 8-CH₂CH₃), 0.19, −2.36 (each br s, 2H, NH); ¹³C NMR (100 MHz, CDCl₃, δ ppm^{#}): 192.80/192.79, 185.3, 174.9, 173.63/173.62, 166.94/166.91, 153.9, 152.7, 151.8, 144.9, 142.0/141.9, 140.53/140.52, 140.21/140.20, 138.1, 136.94, 136.87, 136.4, 133.8/133.7, 132.5/132.4, 130.5, 130.2, 127.2, 126.6, 105.2, 104.6, 102.4, 95.4, 94.5, 72.23/72.21, 70.5, 52.7, 51.6, 49.37/49.36, 31.80/31.79, 31.5, 25.02/24.98, 23.9/23.8, 19.6, 17.6, 12.7, 11.5, 11.2. ^{#1}H and ¹³C deltas calibrated to chloroform resonances at 7.26 ppm and 77.0 ppm, respectively. HR-ESI-MS C₄₁H₄₁N₄O₅I [MH]⁺ calcd 797.2196 found 797.2212, UV-vis (CH₂Cl₂, λₘₐₓ, nm (ϵ)): 386 (8.76×10⁴), 415 (6.38×10⁴), 513 (1.07×10⁴), 672 (5.41×10⁴).

Synthesis of 3-(1′-m-iodobenzyloxy)ethyl-3-devinylverdin (4): Compound 3 (20 mg) was dissolved in DCM (10 mL) and diazomethane, prepared from 400 mg of N-methyl-N-nitroso-p-toluensulfonamide (Diazald), was added. The reaction mixture was kept at room temperature in a sealed flask without stirring in the dark overnight, after which the solvent was evaporated in vacuum. The product was purified by prep-TLC using 4% Acetone in DCM as eluent. 9 mg of 4 was obtained. ¹H NMR (400 MHz, CDCl₃, δ ppm^{#}): 9.49 (s, 1H, 5-H), 9.06 (s, 1H, 10H), 8.31 (s, 1H, 20-H), 7.75 (br s, 1H, phenyl-H), 7.64 (m, 1H, phenyl-H), 7.28 (m, 1H, phenyl-H), 7.061/7.056 (dd, 1H, J=7.8, 7.8 Hz, phenyl-H), 6.94 (s, 1H, 13¹-H), 5.77 (q, 1H, J=6.7 Hz, 3¹-H), 5.02 (dd, 1H, J=2.2, 9.2 Hz, 17-H), 4.66/4.65 (d, 1H, J=11.9 Hz, —OCHH), 4.51/4.50 (d, 1H, J=11.9 Hz, O—CHH), 4.15 (q, 1H, J=7.3 Hz, 18-H), 4.10 (s, 3H, 13²-OCH₃), 3.59/3.58 (s, 3H, —CO₂CH₃), 3.53 (q, 2H, J=7.6 Hz, 8-CH₂CH₃), 3.26 (s, 3H, 12-CH₃), 3.19/3.18 (s, 3H, 2-CH₃), 3.033/3.029 (s, 3H, 7-CH₃), 2.87 (m, 1H, 17-CH₂CHHCO₂CH₃), 2.55 (m, 1H, 17-CH₂CHHCO₂CH₃), 2.29 (m, 1H, 17-CHHCH₂CO₂CH₃), 2.07/2.06 (d, 3H, J=6.7 Hz, 3¹-CH₃), 1.83 (m, 1H, 17-CHHCH₂CO₂CH₃), 1.64 (d, 3H, J=7.3 Hz, 18-CH₃), 1.60 (t, 3H, J=7.6 Hz, 8-CH₂CH₃), 1.26 (br s, 1H, NH), 0.87 (br s, 1H, NH); ¹³C NMR (100 MHz, CDCl₃, δ ppm^{#}): 183.2, 178.0, 174.08/174.06, 171.5, 153.1, 152.1, 151.3, 143.2, 140.8, 140.73/140.71, 140.1, 138.9, 137.0, 136.9, 136.8, 135.1, 132.63/132.55, 132.4, 130.1, 129.31/129.28, 127.18/127.17, 126.4, 106.3, 105.4, 103.7, 103.0, 95.9, 94.5, 71.88/71.86, 70.17/70.16, 56.17/56.15, 55.9, 51.4, 47.8, 33.0, 30.5, 24.70/24.67, 24.07/24.05, 19.3, 17.4, 11.1, 10.77/10.76, 10.6. ^{#1}H and ¹³C deltas calibrated to chloroform resonances at 7.26 ppm and 77.0 ppm, respectively. HR-ESI-MS C₄₃H₄₅N₄O₅I [MH]⁺ calcd 825.2498 found 825.2516, UV-vis (CH₂Cl₂, λₘₐₓ, nm (ϵ)): 363 (4.00×10⁴), 422 (11.9×10⁴, 564 (1.42×10⁴), 772 (2.37×10⁴).

Synthesis of 3-[1′-(m-trimethylstannylbenzyloxy)]ethyl-3-devinylpyropheophorbide-a methyl ester (5): The reaction was performed according to the method described in the literature. To a solution of methyl 3-devinyl-3-{1-(m-iodobenzyloxy)ethyl} pyropheophorbide-a (4) (10 mg) in degassed 1,4-dioxane(5 mL) were added hexamethyldistannane (0.1 mL) and bis(triphenylphosphine)palladium(II) dichloride (2 mg), and the reaction mixture was stirred at 60° C. overnight. After reduced pressure rotary evaporating to dryness, the crude mixture was purified by prep-TLC (Analtech silica, 1000 μm). Hexane:ethyl acetate (70:30) was used as eluent to yield Compound 5. Yield: 9 mg (85%). ¹H NMR (400 MHz, CDCl₃, δ ppm^{#}): 9.51 (s, 1H, 5-H), 9.08 (s, 1H, 10-H), 8.30/8.29 (s, 1H, 20-H), 7.30-7.46 (m, 4H, phenyl H), 6.95 (s, 1H, 13¹-H), 5.80/5.79 (q, 1H, J=6.7 Hz, 3¹-H), 5.01 (dd, 1H, J=2.1, 9.2 Hz, 17-H), 4.74/4.73 (d, 1H, J=11.6 Hz, —OCHH), 4.554/4.547 (d, 1H, J=11.6 Hz, —OCHH), 4.14 (q, 1H, J=7.3 Hz, 18-H), 4.10 (s, 3H, 13²-OCH₃), 3.580/3.578 (s, 3H, —CO₂CH₃), 3.53 (q, 2H, J=7.7 Hz, 8-CH₂CH₃), 3.28 (s, 3H, 12-CH₃), 3.18/3.17 (s, 3H, 2-CH₃), 3.00 (s, 3H, 7-CH₃), 2.87 (ddd, 1H, J=5.4, 11.1, 15.7 Hz, 17-CH₂CHHCO₂CH₃), 2.55 (ddd, 1H, J=4.8, 11.5, 15.7 Hz, 17-CH₂CHHCO₂CH₃), 2.28 (m, 1H, 17-CHHCH₂CO₂CH₃), 2.05/2.04 (d, 3H, J=6.7 Hz, 3¹-CH₃), 1.82 (m, 1H, 17-CHHCH₂CO₂CH₃), 1.63/1.62 (d, 3H, J=7.2 Hz, 18-CH₃), 1.61 (t, 3H, J=7.7 Hz, 8-CH₂CH₃), 1.26 (br s, 1H, NH), 0.89 (br s, 1H, NH), 0.21 (s, 9H, —Sn(CH₃)₃); ¹³C NMR (100 MHz, CDCl₃, δ ppm^{#}): 183.2, 177.96/177.95, 174.11/174.10, 171.5, 153.1, 152.3, 151.3/151.2, 143.2, 142.5, 141.0/140.9, 139.9, 139.39/139.35, 137.6, 136.9, 135.62/135.60, 135.29/135.28, 135.01/135.00, 132.84/132.76, 132.3, 129.3/129.2, 128.32/128.29, 128.09/128.08, 126.2, 106.5, 105.6, 103.7, 103.1, 95.9, 71.52/71.49, 71.4, 56.2/56.1, 55.9, 51.4, 47.8, 33.0, 30.5, 24.74/24.72, 24.07/24.05, 19.3, 17.4, 11.0, 10.8, 10.6, −9.6. ^{#1}H and ¹³C deltas calibrated to chloroform resonances at 7.26 ppm and 77.0 ppm, respectively. HR-ESI-MS C₄₆H₅₄N₄O₅Sn [MH]^{+ calcd} 863.3184 found 863.3215.

Synthesis of ¹²⁴I-analog, Methyl 3-[1′-(m-¹²⁴iodobenzyloxy)ethyl]-3-devinyl-13³-keto-13²methoxycyclohexene (verdin) (6): ¹²⁴Iodine was produced in the SUNY Buffalo South Campus facility via ¹²⁴Te(p,n)¹²⁴I reaction. The ¹²⁴TeO target was irradiated by a 14.1 MeV proton beam and the ¹²⁴I produced was purified by dry distillation. The activity was trapped in 0.1 mL of 0.1 N NaOH. The trimethyl-tin analog, 5, (40 μg) was dissolved in 50 μL of 5% acetic acid in methanol, and 100 μL of 5% acetic acid in methanol was added to dried Na¹²⁴I in another tube. The two solutions were mixed and 10 μL of N-chlorosuccinimide in methanol (1 mg/mL) was added. The reaction mixture was incubated at room temperature for 8 minutes, and the reaction product was purified on an HPLC column (Waters Symmetry C-18 5 μm), eluted with a 95:5 mixture of methanol and water at a flow rate of 1 mL/minute. The output was monitored by UV (254 nm) and radioactivity detectors. The labeled product, 6, was collected and dried. Final product was formulated in 10% ethanol in saline for injection in mice for PET imaging and biodistribution studies. Formulations noted as Pluronic® were formulated in 2% Pluronic® F-127 in saline. Mice were injected with 50 μCi (˜100 μL) for biodistribution, or 100 μCi (˜200 μL) for PET imaging.

Instrumentation: Measurement of fluorescence quantum yields were carried out on a Hamamatsu C9920-0X(PMA-12) U6039-05 fluorescence spectrofluorometer. Femtosecond laser flash photolysis was conducted using a Clark-MXR 2010 laser system and an optical detection system provided by Ultrafast Systems (Helios). The source for the pump and probe pulses were derived from the fundamental output of Clark laser system (775 nm, 1 mJ pulse-1 and fwhm=150 fs) at a repetition rate of 1 kHz. A second harmonic generator introduced in the path of the laser beam provided 412 nm laser pulses for excitation. A 95% of the fundamental output of the laser was used to generate the second harmonic, while 5% of the deflected output was used for white light generation. Prior to generating the probe continuum, the laser pulse was fed to a delay line that provided an experimental time window of 1.6 ns with a maximum step resolution of 7 fs. The pump beam was attenuated at 5 μJ pulse-1 with a spot size of 2 mm diameter at the sample cell where it was merged with the white probe pulse in a close angle (<10°). The probe beam after passing through the 2 mm sample cell was focused on a 200 μm fibre optic cable, which was connected to a CCD spectrograph (Ocean Optics, S2000-UV-vis for visible region and Horiba, CP-140 for NIR region) for recording the time-resolved spectra (450-800 and 800-1400 nm). Typically, 5000 excitation pulses were averaged to obtain the transient spectrum at a set delay time. The kinetic traces at appropriate wavelengths were assembled from the time-resolved spectral data. Nanosecond time-resolved transient absorption measurements were carried out using the laser system provided by UNISOKU Co., Ltd. Measurements of nanosecond transient absorption spectrum were performed according to the following procedure. A mixture solution in a quartz cell (1 cm×1 cm) was excited by a Nd:YAG laser (Continuum SLII-10, 4-6 ns fwhm, λₑₓ=355 nm, 80 mJ pulse⁻¹, 10 Hz). The photodynamics were monitored by continuous exposure to a xenon lamp for visible region and halogen lamp for near-IR region as a probe light and a photomultiplier tube (Hamamatsu 2949) as a detector.

Although the present disclosure has been described with respect to one or more particular examples, it will be understood that other examples of the present disclosure may be made without departing from the scope of the present disclosure.

1. A compound having the following structure: 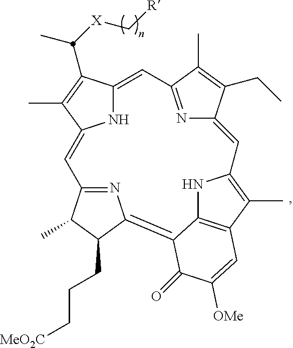
or a salt, a partial salt, a hydrate, a polymorph, an isomer, or a mixture thereof, wherein R′ is an aryl or heteroaryl group having a halogen group, X is chosen from O, S, or NH, n is 1-6, and the dotted carbon is chiral and is R or S.

2. The compound of claim 1, wherein the halogen group is I or ¹²⁴I.

3. The compound of claim 1, wherein the compound has the following structure: 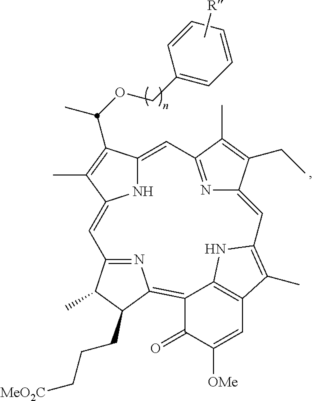
and R″ is a halogen group.

4. The compound of claim 3, wherein the halogen group is I or ¹²⁴I.

5. The compound of claim 3, wherein the compound has the following structure: 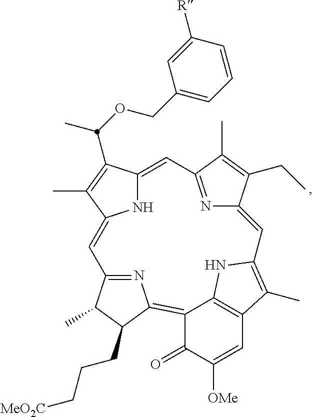
wherein R″ is a halogen group.

6. The compound of claim 5, wherein the halogen group is I or ¹²⁴I.

7. A composition comprising a compound of claim 1 and a pharmaceutically acceptable carrier.

8. A method of imaging a hyperproliferative tissue in an individual, comprising:
administering to the individual one or more compound(s) of claim 1;
exposing the individual or a portion thereof with electromagnetic radiation; and
imaging the individual or a portion thereof.

9. The method of claim 8, wherein after a single administration of the one or more compound(s), imaging is performed up to 24 hours later, up to 48 hours later, up to 72 hours later, and/or up to 96 hours later.

10. The method of claim 8, wherein the hyperproliferative tissue is vascular endothelial tissue, a neovasculature tissue, a neovasculature tissue present in the eye, an abnormal vascular wall of a tumor, a solid tumor, a tumor of the pancreas, a tumor of the bladder, a tumor of a head, a tumor of a neck, a tumor of an eye, a tumor of a gastrointestinal tract, a tumor of a liver, a tumor of a breast, a tumor of a prostate, a tumor of a brain, a tumor of a lung, a nonsolid tumor, malignant cells of one of a hematopoietic tissue, a lymphoid tissue, or combinations thereof.

11. The method of claim 8, wherein the one or more compound(s) is administered as a composition comprising a pharmaceutically acceptable carrier.

12. The method of claim 8, wherein the compound has the following structure: 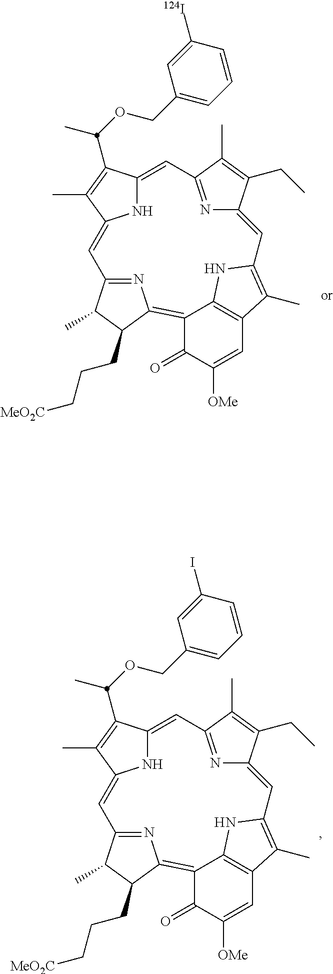 [SMILES: CCC1=C(C)c2cc3[nH]c(cc4nc(c5c6[nH]c(cc1n2)c(C)c6C=C(OC)C5=O)[C@@H](CCCC(=O)OC)[C@@H]4C)c(C)c3[C@H](C)OCc1cccc([124I])c1 ; CCC1=C(C)c2cc3[nH]c(cc4nc(c5c6[nH]c(cc1n2)c(C)c6C=C(OC)C5=O)[C@@H](CCCC(=O)OC)[C@@H]4C)c(C)c3[C@H](C)OCc1cccc(I)c1]
where the dotted carbon is chiral and is R or S.

13. A kit, comprising
one of more compound(s) of claim 1; and
instructions for use of the one or more compound(s).

14. The kit of claim 13, wherein the one or more compound(s) is present in a composition comprising a pharmaceutically acceptable carrier.

15. The kit of claim 13, further comprising a pharmaceutically acceptable carrier.
